(19) United States
(12) Patent Application Publication
(10) Pub. No.: US 2026/0057293 A1
(43) Pub. Date: Feb. 26, 2026

(54) DYNAMIC ADAPTIVE LEARNING METHOD FOR MINERAL PREDICTION, SYSTEM, DEVICE AND MEDIUM THEREFOR

(71) Applicant: Institute of Geology and Geophysics Chinese Academy of Sciences, Beijing (CN)

(72) Inventors: Pengfei Lv, Beijing (CN); Weiying Chen, Beijing (CN); Guoqiang Xue, Beijing (CN)

(21) Appl. No.: 18/950,173

(22) Filed: Nov. 18, 2024

(30) Foreign Application Priority Data
Aug. 21, 2024 (CN) 202411152960.X

(51) Int. Cl. G06N 20/00; G16C 60/00

(52) U.S. Cl. CPC G06N 20/00; G16C 60/00

(57) ABSTRACT

A dynamic adaptive learning method for mineral prediction includes: collecting a dataset including geological data and labels of the geological data; extracting features from the geological data, initializing parameters of a training model and optimizing the parameters to obtain training parameters; performing an associative training on the training model based on the training parameters and the labels in a dynamic adaptive learning framework to obtain a mineral prediction model, algorithms of the associative training including a variational expectation algorithm and a variational maximization algorithm, and the variational expectation algorithm including an unsupervised learning mode, a semi-supervised learning mode, and a fully supervised learning mode; and predicting, by using the mineral prediction model, a mineral to obtain a mineral prediction result. The method can break through limitations of the traditional machine learning technology, offering a more efficient, universal, and stable strategy for geophysical data analysis and mineral resource assessment.

### TECHNICAL FIELD

The disclosure relates to the field of geophysical exploration technologies, and particularly to a dynamic adaptive learning method, system, device and medium for mineral prediction.

### BACKGROUND

In the fields of geological exploration and geophysics, the identification and prediction of target rock masses are crucial. Traditional rock mass identification methods mainly rely on the experience of geologists and simple statistical data processing techniques. These methods often have limited effects when dealing with complex geological data, especially when facing large-scale and multi-source heterogeneous data. With the development of detection technology, both the quantity and quality of detection data have been significantly improved. The detection data not only exhibit multi-source features but also show high dimensionality and complexity. The detection data include, but are not limited to, seismic data, geomagnetic data, gravity data, and various geological and geochemical exploration data. Each type of data carries unique information about the underground structure. However, how to effectively integrate and analyze these multi-source detection data to extract useful information for accurately predicting the location and properties of rock masses remains a huge challenge.

Currently, machine learning technology has shown strong data processing capabilities in various fields, especially in image recognition and natural language processing, where breakthrough progress has been made. This has inspired researchers to apply the machine learning technology to geological data analysis in the hope of solving problems that traditional technology cannot overcome. Although the machine learning technology provides new possibilities for data analysis, designing an effective learning framework that adapts to the features of geological data in practical applications is still a technical challenge.

However, despite the significant advantages of data-driven methods based on the machine learning technology in dealing with complex geological data and identifying potential mineral resources, they often lack universality and adaptability in multi-mineral areas or different sections of complex mining areas with multi-data environments. On the one hand, there are significant differences in the data sets collected from different mining areas, and many existing methods mainly focus on an application of single data scenarios. On the other hand, as the exploration of the mining areas becomes increasingly complex and the exploration range continues to expand, the data sources collected from the different mining areas are quite diverse. At this time, there will be a mismatch in data features, such as vegetation-covered areas often using an aviation method or a semi-aviation detection method, and flat terrain areas are more inclined to use a ground detection method. In addition, due to the aggregation and uneven distribution of ore deposits in space, acquisitions of data labels will also be unbalanced. Using the single machine learning technology at a scale structure of the mining area will lead to regional imbalance and information asymmetry in model training, thereby affecting the generalization performance of the model.

### SUMMARY

The purpose of the disclosure is to provide a dynamic adaptive learning method, system, device and medium for mineral prediction, which can break through limitations of the traditional machine learning technology, offering a more efficient, universal, and stable strategy for geophysical data analysis and mineral resource assessment.

To achieve above purpose, the technical solutions are as follows.

A dynamic adaptive learning method for mineral prediction includes the steps as follows.

A dataset is collected, and the dataset includes geological data and labels of the geological data.

Features are extracted from the geological data to obtain extracted features, parameters of a training model are initialized, and the parameters of the training model are optimized to obtain trained parameters, and then associative training is performed on the training model based on the extracted features, the training parameters and the labels in a dynamic adaptive learning framework to obtain a mineral prediction model. Algorithms of the associative training include a variational expectation algorithm and a variational maximization algorithm, and the variational expectation algorithm includes an unsupervised learning mode, a semi-supervised learning mode, and a fully supervised learning mode.

A mineral is predicted by using the mineral prediction model to obtain a mineral prediction result.

In an embodiment, the mineral prediction result is configured to obtain a distribution of a target mineral resource layer in a mining area, thereby to locate and mine mineral resources in the target mineral resource layer based on the distribution of the target mineral resource layer; and the distribution of the target mineral resource layer includes an elevation range of the target mineral resource layer.

In an embodiment, the labels include 0 and non-0, where 0 represents non-lithology, and the non-0 represents different lithologies.

In an embodiment, the performing associative training on the training model based on the extracted features, the training parameters and the labels in a dynamic adaptive learning framework includes: inputting the trained parameters into the dynamic adaptive learning framework to construct the training model; and inputting the extracted features into the training model, and performing supervised learning training on the training model with a goal of minimizing a loss between model output results and the labels to obtain a trained model as the mineral prediction model.

In an embodiment, the associative training includes: using the unsupervised learning mode, in response to each of the labels being 0; using the semi-supervised learning mode, in response to the labels including 0 and non-0; and using the fully supervised learning mode, in response to each of the labels being non-0.

In an embodiment, after obtaining the mineral prediction model, the method further includes performing model evaluation and geological interpretation. The model evaluation includes performing evaluation by using a silhouette coefficient distribution, in response to using the unsupervised learning mode; and performing the evaluation by using a calculation accuracy, a confusion matrix function, and a F1 function, in response to using the semi-supervised learning mode or the fully supervised learning mode.

A dynamic adaptive learning system for mineral prediction is provided, and includes a dataset generating unit, a model training unit and a model predicting unit, and the dataset generating unit, the model training unit and the model predicting unit each are embodied by software stored in at least one memory and executable by at least one processor.

The dataset generating unit is configured to collect a dataset, and the dataset includes geological data and labels of the geological data. The model training unit is configured to extract features from the geological data to obtain extracted features, initialize parameters of a training model and optimize the parameters of the training model to obtain trained parameters, and perform associative training on the training model based on the extracted features, the trained parameters and the labels in a dynamic adaptive learning framework to obtain a mineral prediction model, algorithms of the associative training include a variational expectation algorithm and a variational maximization algorithm, and the variational expectation algorithm includes an unsupervised learning mode, a semi-supervised learning mode, and a fully supervised learning mode. The model predicting unit is configured to predict a mineral by using the mineral prediction model to obtain a mineral prediction result.

An electronic device includes a processor and a memory. The memory stores a computer program executable by the processor, and the processor is configured to execute the computer program to implement the above method.

A computer-readable storage medium is provided, the computer-readable storage medium stores a computer program, and the computer program is configured to be executed by a processor to implement the above method. The computer-readable storage medium may be a non-transitory computer-readable storage medium.

The technical effects of the disclosure are as follow.

The disclosure provides a dynamic adaptive learning method, system, device and medium for mineral prediction. The method includes collecting a dataset including geological data and labels of the geological data; extracting features from the geological data to obtain extracted features, initializing parameters of a training model and optimizing the parameters of the training model to obtain training parameters, and then performing associative training on the training model based on the extracted features, the training parameters and the labels in a dynamic adaptive learning framework to obtain a mineral prediction model; where the algorithms of the associative training include a variational expectation algorithm and a variational maximization algorithm, and the variational expectation algorithm includes an unsupervised learning mode, a semi-supervised learning mode, and a fully supervised learning mode; and predicting, by using the mineral prediction model, a mineral to obtain a mineral prediction result.

The disclosure can break through the limitations of traditional machine learning technology, providing a more efficient, universal, and stable strategy for geophysical data analysis and mineral resource assessment.

### BRIEF DESCRIPTION OF DRAWINGS

In order to more clearly illustrate the embodiments of the disclosure or the technical solutions in the related art, a brief introduction will be given to the attached drawings required for the embodiments. It is apparent that the attached drawings are only some embodiments of the disclosure. For those skilled in the art, other drawings can be obtained based on the attached drawings without creative labor.

FIG. 1 illustrates a flowchart of a dynamic adaptive learning algorithm for mineral prediction of the disclosure.

FIG. 2A illustrates a schematic diagram of a three-dimensional (3D) display of a grid density in an embodiment of the disclosure.

FIG. 2B illustrates a schematic diagram of the 3D display of a resistivity in the embodiment of the disclosure.

FIG. 3 illustrates a schematic diagram of drilling verification results in the embodiment of the disclosure.

FIG. 4A illustrates a schematic diagram of a 3D mineral prediction model and geological interpretation results in the embodiment of the disclosure.

FIG. 4B illustrates a perspective view of the target.

### DETAILED DESCRIPTION OF EMBODIMENTS

The following will combine the attached drawings in the embodiment of the disclosure to provide a clear and complete description of the technical solution in the embodiment of the disclosure. Apparently, the embodiment is only a part of the embodiments of the disclosure, not all of them. Based on the embodiment in the disclosure, all other embodiments obtained by those skilled in the art without creative labor are within the scope of protection of the disclosure.

The purpose of the disclosure is to provide a dynamic adaptive learning method, system, device and medium for mineral prediction, which can break through limitations of the traditional machine learning technology, offering a more efficient, universal, and stable strategy for geophysical data analysis and mineral resource assessment.

In order to make the above objectives, features, and advantages of the disclosure more obvious and understandable, the following will provide further detailed explanations of the disclosure in conjunction with the attached drawings and specific embodiments.

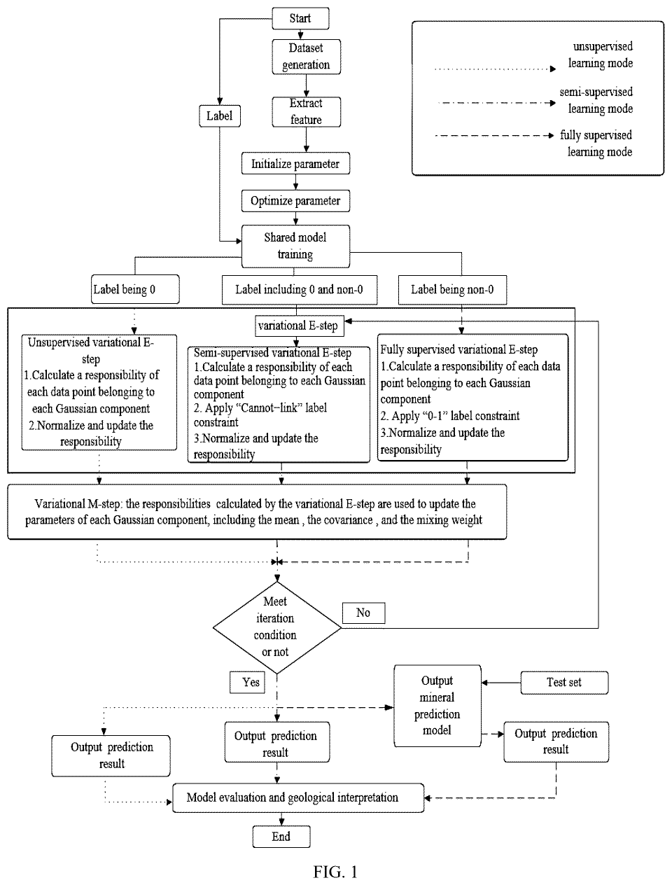

As shown in FIG. 1, a dynamic adaptive learning method for mineral prediction is provided and includes steps as follows.

Step 100: a dataset is collected, and the dataset includes geological data and labels of the geological data.

Step 200: features are extracted from the geological data, parameters of a training model are initialized, and the parameters are optimized, to obtain trained parameters, then associative training is performed on the training model based on the extracted features, the trained parameters and the labels in a dynamic adaptive learning framework to obtain a mineral prediction model. Algorithms of the associative training include a variational expectation algorithm and a variational maximization algorithm, and the variational expectation algorithm includes an unsupervised learning mode, a semi-supervised learning mode, and a fully supervised learning mode.

Step 300: a mineral is predicted by using the mineral prediction model to obtain a mineral prediction result.

### Embodiment 1

Based on above technical solutions, the method includes the steps as follows. [0038](1) Dataset generation: geophysical data, geochemical data, remote sensing data, hyperspectral data, and other data are integrated into the same coordinate system to form a feature set (Features) of the data samples. Data (lithology) provided by prior geological information from drilling are used as labels, and followed by matching the labels to the same position in the feature set. Corresponding labels are designed for different lithologies represented by non-0 (i.e., non-zero numbers), and the labels with non-lithology are represented by 0. The dataset is shown in Table 1. After the dataset is generated, quasi-Gaussian and normalization processing are performed on the feature set to update the dataset.

TABLE 1
dataset

| X | D | Resistivity | Density | Features | Velocity | Lithology | Label |
|---|---|---|---|---|---|---|---|
| 1650 | 690 | 417.600 | −0.143 | . . . | 2.903 | Siltstone | 1 |
| 1650 | 680 | 406.973 | −0.146 | . . . | 2.899 | Siltstone | 1 |

. . .

| | | | | | | | |
|---|---|---|---|---|---|---|---|
| 1650 | 670 | 397.575 | −0.150 | . . . | 2.895 | Siltstone | 1 |
| 950 | 1010 | 4901.917 | 0.394 | . . . | 3.021 | Mineralized | 2 |
| | | | | | | dolomite | |
| 950 | 1000 | 4847.334 | 0.396 | . . . | 3.023 | Mineralized | 2 |
| | | | | | | dolomite | |

. . .

| | | | | | | | |
|---|---|---|---|---|---|---|---|
| 950 | 980 | 4685.010 | 0.400 | . . . | 3.029 | Mineralized | 2 |
| | | | | | | dolomite | |
| 1650 | 530 | 2185.806 | 0.381 | . . . | 2.915 | Slate | 3 |
| 1650 | 520 | 2188.655 | 0.371 | . . . | 2.917 | Slate | 3 |

. . .

| | | | | | | | |
|---|---|---|---|---|---|---|---|
| 1650 | 510 | 2170.739 | 0.360 | . . . | 2.919 | Slate | 3 |
| 1250 | 890 | 72.016 | 0.489 | . . . | 2.280 | Dolomite | 4 |
| 1250 | 980 | 63.449 | 0.492 | . . . | 2.236 | Dolomite | 4 |

. . .

| | | | | | | | |
|---|---|---|---|---|---|---|---|
| 1250 | 970 | 67.813 | 0.490 | . . . | 2.258 | Dolomite | 4 |
| 0 | 1700 | 75.023 | 0.115 | . . . | 1.985 | Unknown | 0 |
| 0 | 1690 | 77.176 | 0.114 | . . . | 1.987 | Unknown | 0 |

. . .

| | | | | | | | |
|---|---|---|---|---|---|---|---|
| 0 | 1680 | 79.812 | 0.114 | . . . | 1.989 | Unknown | 0 |
| 0 | 1670 | 82.905 | 0.114 | . . . | 1.991 | Unknown | 0 |

(2) Extracting features: a MATLAB dimensionality reduction toolbox is installed, and a most suitable dimensionality reduction tool is independently selected from the MATLAB dimensionality reduction toolbox to provide optimized data input for a subsequent model training.
(3) Initializing parameters: before the model training, it is necessary to preliminarily estimate parameters of a training model to obtain initialized parameters of the training model, which includes a mean μₖ, a covariance Λₖ, and a mixing weight πₖ of each Gaussian component.
(4) Optimizing the parameters: the purpose of the step is global parameter optimization to improve the efficiency of hyperparameter tuning. This process selects πₖ and β as optimization targets and designs a genetic algorithm function. When the algorithms run, it first sets ranges of πₖ and β, a number of iterations for the genetic algorithm function, and convergence conditions, where πₖ represents the mixing weight, and β represents a precision parameter of the Gaussian distribution related to a matrix of the covariance Λₖ. GA starts with a set of randomly generated candidate solutions, and in each generation, it selects the individuals most adapted to the environment for genetic operations to generate a new generation. This process is repeated until a termination condition is met, and the optimized parameters of πₖ and β are output.
(5) Dynamic adaptive model training: the dataset generated in step (1), the optimized parameters obtained in step (4), and other initialized parameters from step (3) are input into a designed dynamic adaptive learning framework. By associating labels with learning modes, the dynamic adaptive learning on the dataset is realized, enhancing a predictive ability of the model. The algorithm (i.e., variational EM algorithm) alternately executes two algorithm steps: a variational expectation algorithm step (E-step) and a variational maximization algorithm step (M-step).

The variational E-step: first, a posterior probability (i.e., a responsibility γᵢⱼ) of each data point xᵢ belonging to each Gaussian component is calculated based on the optimized parameters of πₖ and β obtained in the step (4) and other parameters from step (3), and formula (1) is shown as follows:

$${γ}_{ij} = E [ ln {π}_{k} ] + \frac{1}{2} E [ ln \vert {Λ}_{k} \vert ] - \frac{D}{2} ln ( 2 π ) - \frac{1}{2} {E}_{{μ}_{k} , {Λ}_{k}} [ {( {x}_{n} - {μ}_{k} )}^{T} {Λ}_{k} ( {x}_{n} - {μ}_{k} ) ] \qquad ( 1 )$$

where πₖ represents a mixing weight of a k-th Gaussian component, μₖ represents a mean of a k-th Gaussian component, Λₖ represents a covariance of a k-th Gaussian component, and rᵢⱼ represents a responsibility of a data point i belonging to a distribution j.

However, different label situations correspond to different learning modes. When each of the labels is 0, an unsupervised learning mode is selected. When the labels include 0 and non-0, a semi-supervised learning mode is selected. When each of the labels is non-0, a fully supervised learning mode is selected. Subsequently, the responsibility matrix γᵢⱼ is processed flexibly according to the learning modes.

Unsupervised learning mode (A): normalization of the responsibility γᵢⱼ for each data point as shown in formula (2) is required to ensure that a sum of the responsibilities for each data point equals 1:

$${γ}_{ij}^{UL} = \frac{{γ}_{ij}}{{\sum }_{j = 1}^{k} {γ}_{ij}} \qquad ( 2 )$$

Semi-supervised learning mode (B): after normalizing the responsibility rᵢⱼ for each data point to obtain

$${γ}_{{1}_{ij}^{SsL}} ,$$

a “Cannot-link” label constraint is applied. First, its most likely cluster j for each labeled data point xᵢ is need to be found, as shown in formula (3):

$${j}^{*} ( i ) = arg {max}_{j} {{γ}^{1}}_{ij} \qquad ( 3 )$$

where j*(i) represents an index of the most likely cluster for the data point xᵢ. For each cluster j and each data point xᵢ, if an actual label l(i) of xᵢ is different from the label l(j*) of the representative data point of the cluster j, the responsibility of data point xᵢ belonging to the cluster j is reduced to update, and as shown in formula (4):

$${γ}_{{2}_{ij}^{SsL}} = \{ \begin{matrix}{εγ}_{{1}_{ij}^{SsL}} & if l ( i ) \neq l ( {j}^{*} ) \\ {γ}_{{1}_{ij}^{SsL}} & otherwise\end{matrix} \qquad ( 4 )$$

the updated responsibilities are normalized to ensure that a sum of responsibilities for all clusters for each data point equals 1, and the responsibility matrix

$${γ}_{{3}_{ij}^{SsL}}$$

of the training model is finally updated.

Fully supervised learning mode (C): according to the following formula (5), the responsibility of the data point is directly set to ensure that it is fully assigned to the cluster specified by its label:

$${γ}_{ij}^{SL} = \{ \begin{matrix}1 & if j = l ( i ) \\ 0 & otherwise\end{matrix} \qquad ( 5 )$$

where

$${γ}_{ij}^{SL}$$

is the responsibility of assigning data point xᵢ to the cluster j, and l(i) is an actual label of data point xᵢ.

The variational M-step: the responsibilities γ^{new}ᵢⱼ calculated by the variational E-step are used to update the parameters of each Gaussian component, including the mean μₖ, the covariance Λₖ, and the mixing weight πₖ. The parameter update can be achieved by maximizing the expected complete data log-likelihood obtained in the variational E-step.

The variational EM algorithm iterates through the variational E-step and the variational M-step until convergence. The convergence is determined based on the change in the variational lower bound (ELBO). When the increase in ELBO is less than a preset threshold, the variational EM algorithm is considered to have converged.
(6) Mineral prediction result output: first, based on the situation of the ELBO, it is determined whether the variational EM algorithm has converged. If the ELBO no longer increases, the variational EM algorithm is considered stable and converged. Subsequently, an output of the prediction result is determined. For the unsupervised learning mode, the output of the prediction result is the mineral prediction result. For the semi-supervised learning mode, the output of result for the data originally labeled as 0 is the mineral prediction result. However, for supervised learning, after the model training is completed, only the mineral prediction model is obtained. It is necessary to prepare the training set and input it into the mineral prediction model to calculate the probabilities of the training set. By identifying the cluster index with the highest probability for each data point, a predicted category for each data point is determined.
(7) Model evaluation and geological interpretation: for the unsupervised learning mode, a silhouette coefficient distribution is used to evaluate a model performance; for the semi-supervised learning and fully supervised learning modes, a calculation accuracy, a confusion matrix function, and a F1 function are used to evaluate the model performance. Finally, the mineral prediction results are subjected to the geological interpretation and compared with other prior geological information.

### Embodiment 2

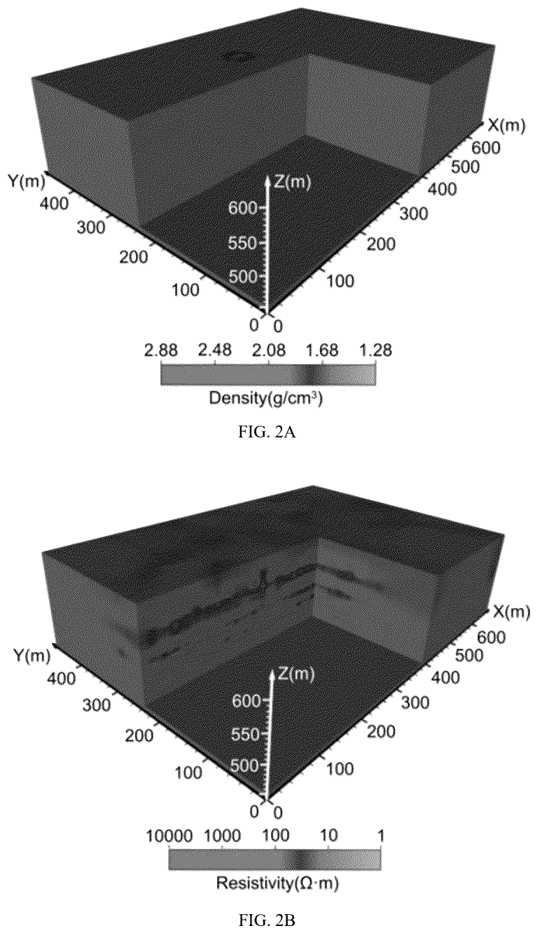

In the embodiment, the experimental data utilizes the geophysical data and the drilling data from the “density-resistivity-efb.intermediate” example in the Earth Volumetric Studio (EVS) 3D geological modeling software's built-in library. This dataset includes a stratum density data and a resistivity data within a certain depth range of a survey area, and a drilling data from 22 bore holes. The main objective of the dataset is to identify coal seams based on the density and resistivity features of the strata. In the embodiment, the density data and the resistivity data are first gridded using the Kriging grid algorithm with a dimension of 5 meters (m) by 5 m by 5 m in three directions, and then imaged using Surfer software as shown in FIG. 2A and FIG. 2B. In the FIG. 2A and FIG. 2B, a portion of the data is removed to better display the distribution of the internal data. For the drilling data, the embodiment lists the positional coordinates and revealed stratigraphic information in Table 2.

First, the geophysical data and the drilling data are unified under the same coordinate system to generate the required dataset. The dataset contains a total of 34,125 data entries, of which 897 have label data. In the dataset, the bore holes of BC-725 and BC-719 are used as validation holes and are not used for the model training. The mining area is a typical coal seam mining area, and according to the drilling data, there is a clear stratification between the media in this area, which is mainly divided into four types of media. Among them, medium 3 is the target of the embodiment, which is the coal seam. In the embodiment, the known labeled data is marked with Arabic numerals 1-4 according to the media type, and unlabeled data is marked with “0”.

After the dataset is generated, depth, density, and resistivity are used as the features for the model training and pre-processing operations such as normalization are performed on the features. Considering that the labeled data only accounts for 2% of the total data, to ensure the reliability of the prediction results, both labeled and unlabeled data are input into the model. The entire dataset is first input into the MS-DALF, and since the labels contain 0, semi-supervised learning mode is automatically performed. The search range for parameters is set from 0.01 to 10, PopulationSize is set to a population size of 50, and MaxGenerations is set to a maximum of 50 iterations. At this point, the genetic algorithm finds the optimal Alpha and kappa parameter values to be 0.1 and 0.096, respectively. Subsequently, the optimal parameters are input into the SsL-VGMM for the model training. After 216 iterations, the model converges, and no new categories are found in the obtained prediction results.

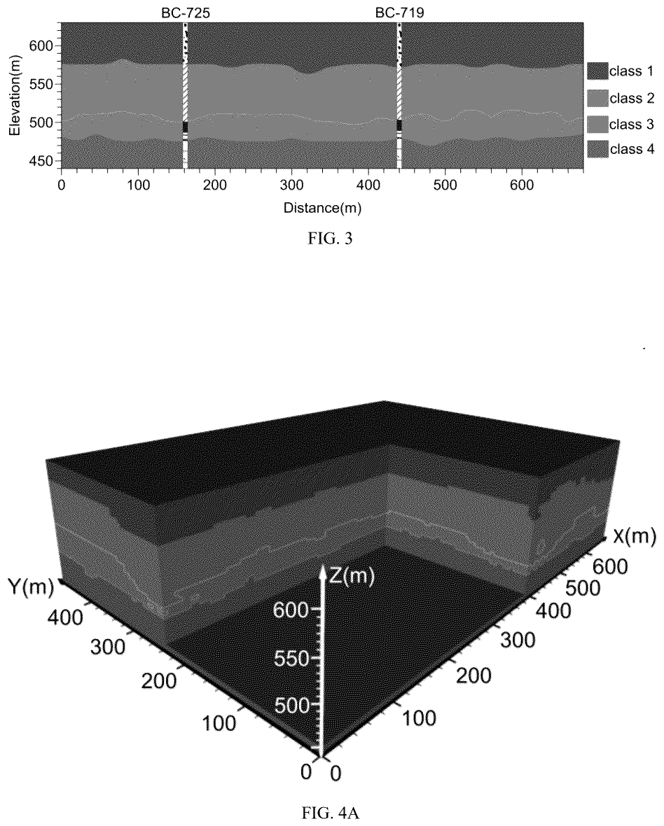

After the model training is completed, a classification accuracy of the labeled data is 100%. Furthermore, by comparing the validation drilling holes of BC-725 and BC-719 with the predicted results of the embodiment, as shown in FIG. 3, it is found that the agreement between different rock types and the predicted results of the embodiment is 100%. This further demonstrates the reliability of the method proposed in the embodiment.

TABLE 2
Stratigraphic information list

| Boreholes | X | Y | TOP | Boundary1 | Boundary2 | Boundary3 | Bottom |
|---|---|---|---|---|---|---|---|
| BC-709 | 597.01 | 182.43 | 621.98 | 586.31 | 514.28 | 484 | 440 |
| BC-710 | 428.82 | 160.48 | 624.98 | 583.18 | 513 | 482.68 | 440 |
| BC-711 | 276.2 | 80.61 | 623.72 | 586.36 | 513.45 | 484 | 440 |
| BC-712 | 156.26 | 52.88 | 621.94 | 586.24 | 512 | 482.81 | 440 |
| BC-713 | 654.97 | 265.15 | 623.67 | 584.67 | 513.72 | 487.32 | 440 |
| BC-714 | 498.84 | 229 | 625.66 | 586.96 | 513.18 | 483.39 | 440 |
| BC-715 | 334.63 | 177.89 | 625.24 | 587.54 | 512.55 | 483.5 | 440 |
| BC-716 | 201.54 | 137.59 | 623.38 | 585.91 | 513.31 | 483.2 | 440 |
| BC-717 | 86.64 | 117.24 | 620.82 | 584.44 | 512.12 | 482.83 | 440 |
| BC-718 | 545.3 | 316.88 | 627.07 | 586.07 | 517.95 | 487.96 | 440 |
| BC-719 | 414 | 278.8 | 627.3 | 583.84 | 513.01 | 482.54 | 440 |
| BC-720 | 244.27 | 226.41 | 624.71 | 585.09 | 512.71 | 483.52 | 440 |
| BC-721 | 111.07 | 189.93 | 621.64 | 585.1 | 512.34 | 482.67 | 440 |
| BC-722 | 556.17 | 399.2 | 629.08 | 590.52 | 518.2 | 487.89 | 440 |
| BC-723 | 458.46 | 364.97 | 629 | 585.8 | 518.04 | 487.88 | 440 |
| BC-724 | 290.34 | 319.58 | 626.69 | 585.24 | 514.29 | 483.89 | 440 |
| BC-725 | 157.45 | 277.13 | 623.28 | 585.81 | 513.07 | 483.92 | 440 |
| BC-726 | 33.34 | 208.08 | 619.3 | 585.96 | 511.84 | 482.4 | 440 |
| BC-727 | 83.42 | 297.06 | 621.37 | 583.84 | 513.16 | 484.43 | 440 |
| BC-728 | 195.37 | 352.45 | 625.4 | 586.98 | 513.91 | 484.15 | 440 |
| BC-729 | 359.53 | 396.72 | 628.62 | 587.84 | 517.66 | 487.67 | 440 |
| BC-730 | 500.84 | 442.28 | 630.54 | 590.21 | 518.34 | 488.43 | 440 |

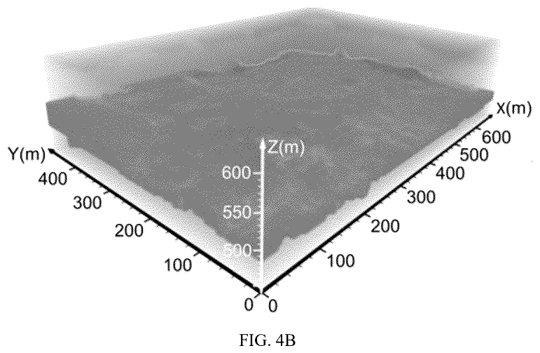

FIG. 4A and FIG. 4B show the predicted results for the entire mining area. A portion has been removed to better display the distribution of the internal data in FIG. 4A and FIG. 4B. It can be observed that there are four distinct layers within the mining area. The third layer is the coal seam, which is also the target layer. The coal seam is primarily distributed in the elevation range of 480 m to 510 m, with a thickness of approximately 30 m. This result is consistent with the actual conditions of the survey area and is more scientific and reliable compared to the traditional method based on the estimation of the range of physical property parameters.

Therefore, to address the problems in the related art, the disclosure has developed an innovative dynamic adaptive learning framework-MS-DALF, which is characterized by high modularity and the ability to more flexibly adapt to differences between mining areas, such as varying geological backgrounds and exploration technologies, thereby enhancing the accuracy of prediction and analysis. The dynamic adaptive learning framework uses the labels of the dataset as a “handle” for the dynamic transformation of data scenarios, employs a variational Gaussian mixture model as a shared model, and designs proprietary learning methods and evaluation criteria for different data scenarios, ensuring the model's efficiency and precision in training and prediction across various tasks. Moreover, the feature dimension reduction module of the MS-DALF framework can flexibly select feature dimension reduction tools, effectively alleviating the issue of mismatched feature sets between different mining sections. The disclosure aims to break through the limitations of traditional machine learning methods and provide a more efficient, universal, and stable strategy for geophysical data analysis and mineral resource assessment. With the aid of the algorithms in the disclosure, it is possible to more accurately identify and locate mineral resources, optimize the allocation and application of exploration resources, which is particularly important and valuable in future explorations.

In the specification, each embodiment is described in a progressive manner, with each embodiment focusing on its differences from the others. For the similar parts between embodiments, cross-referencing is sufficient.

Concrete examples are used in the disclosure to illustrate the principles and embodiment of the disclosure. The descriptions of the above embodiments are provided solely for the purpose of aiding in the understanding of the core concepts of the disclosure. In addition, for those skilled in the art, there will be variations in the specific disclosure and the scope of disclosure based on the ideas of the disclosure. In summary, the content of the specification should not be understood as limiting the disclosure.

What is claimed is:

1. A dynamic adaptive learning method for mineral prediction, comprising:
collecting a dataset, wherein the dataset comprises geological data and labels of the geological data;
extracting features from the geological data to obtain extracted features, initializing parameters of a training model and optimizing the parameters of the training model to obtain training parameters, and then performing associative training on the training model based on the extracted features, the training parameters and the labels in a dynamic adaptive learning framework to obtain a mineral prediction model, wherein algorithms of the associative training comprise a variational expectation algorithm and a variational maximization algorithm, and the variational expectation algorithm comprises an unsupervised learning mode, a semi-supervised learning mode, and a fully supervised learning mode; and
predicting, by using the mineral prediction model, a mineral to obtain a mineral prediction result.

2. The dynamic adaptive learning method for the mineral prediction as claimed in claimed 1, wherein the labels comprise 0 and non-0, where 0 represents non-lithology, and the non-0 represents different lithologies.

3. The dynamic adaptive learning method for the mineral prediction as claimed in claimed 1, wherein the performing associative training on the training model based on the extracted features, the training parameters and the labels in a dynamic adaptive learning framework comprises:
inputting the training parameters into the dynamic adaptive learning framework to construct the training model; and
inputting the extracted features into the training model, and performing supervised learning training on the training model with a goal of minimizing a loss between model output results and the labels to obtain a trained model as the mineral prediction model.

4. The dynamic adaptive learning method for the mineral prediction as claimed in claimed 2, wherein the associative training comprises:
using the unsupervised learning mode, in response to each of the labels being 0;
using the semi-supervised learning mode, in response to the labels including 0 and non-0; and
using the fully supervised learning mode, in response to each of the labels being non-0.

5. The dynamic adaptive learning method for the mineral prediction as claimed in claimed 1, after obtaining the mineral prediction model, further comprising:
performing model evaluation and geological interpretation;
wherein the model evaluation comprises:
performing evaluation by using a silhouette coefficient distribution, in response to using the unsupervised learning mode; and
performing evaluation by using a calculation accuracy, a confusion matrix function, and a F1 function, in response to using the semi-supervised learning mode or the fully supervised learning mode.

6. A dynamic adaptive learning system for mineral prediction, comprising:
a dataset generating unit, configured to collect a dataset, wherein the dataset comprises geological data and labels of the geological data;
a model training unit, configured to extract features from the geological data to obtain extracted features, initialize parameters of a training model and optimize the parameters of the training model to obtain trained parameters, and perform associative training on the training model based on the extracted features, the trained parameters and the labels in a dynamic adaptive learning framework to obtain a mineral prediction model, wherein algorithms of the associative training comprise a variational expectation algorithm and a variational maximization algorithm, and the variational expectation algorithm comprises an unsupervised learning mode, a semi-supervised learning mode, and a fully supervised learning mode; and
a model predicting unit, configured to predict a mineral by using the mineral prediction model to obtain a mineral prediction result.

7. An electronic device, comprising: a processor and a memory; wherein the memory stores a computer program executable by the processor, and the processor is configured to execute the computer program to implement the method as claimed in claim 1.

8. A non-transitory computer-readable storage medium, storing a computer program, wherein the computer program is configured to be executed by a processor to implement the method as claimed in claim 1.
